(19) United States
(12) Patent Application Publication
(10) Pub. No.: US 2023/0402486 A1
(43) Pub. Date: Dec. 14, 2023

(54) IMAGING METHODS USING RADIATION DETECTORS

(71) Applicant: SHENZHEN XPECTVISION TECHNOLOGY CO., LTD., Shenzhen (CN)

(72) Inventors: Peiyan CAO, Shenzhen (CN); Yurun LIU, Shenzhen (CN)

(21) Appl. No.: 18/238,085

(22) Filed: Aug. 25, 2023

Related U.S. Application Data
(63) Continuation of application No. PCT/CN2021/079177, filed on Mar. 5, 2021.

(51) Int. Cl. H01L 27/146

(52) U.S. Cl. CPC H01L 27/14661; H01L 27/14634; H01L 27/14636

(57) ABSTRACT

Disclosed herein is a method, comprising: capturing via an exposure a first image with a first radiation detector which comprises a first active area and a first dummy area, wherein the first dummy area is disposed between application-specific integrated circuit (ASIC) chips of the first radiation detector, and wherein the first image comprises (A) first regular picture elements corresponding to the first active area and (B) first dummy picture elements corresponding to the first dummy area; and determining values of the first dummy picture elements based on values of the first regular picture elements.

### TECHNICAL FIELD

The disclosure herein relates to imaging methods using radiation detectors.

### BACKGROUND

A radiation detector is a device that measures a property of a radiation. Examples of the property may include a spatial distribution of the intensity, phase, and polarization of the radiation. The radiation may be one that has interacted with an object. For example, the radiation measured by the radiation detector may be a radiation that has penetrated the object. The radiation may be an electromagnetic radiation such as infrared light, visible light, ultraviolet light, X-ray or γ-ray. The radiation may be of other types such as α-rays and β-rays. An imaging system may include multiple radiation detectors.

### SUMMARY

Disclosed herein is a method, comprising: capturing via an exposure a first image with a first radiation detector which comprises a first active area and a first dummy area, wherein the first dummy area is disposed between application-specific integrated circuit (ASIC) chips of the first radiation detector, and wherein the first image comprises (A) first regular picture elements corresponding to the first active area and (B) first dummy picture elements corresponding to the first dummy area; and determining values of the first dummy picture elements based on values of the first regular picture elements.

In an aspect, the method further comprises assigning the determined values to the first dummy picture elements.

In an aspect, the first dummy area comprises K straight strips parallel to each other, and wherein K is a positive integer.

In an aspect, a mask blocks any or almost any radiation particle of the exposure that is (A) not aimed at the first radiation detector or (B) aimed at a gutter ring of the first radiation detector.

In an aspect, the first dummy area comprises multiple dummy sensing elements each of which comprises an electrical contact which is (A) other than a same common electrical contact shared by the multiple dummy sensing elements, and (B) not electrically connected to the ASIC chips.

In an aspect, the first dummy area comprises multiple dummy sensing elements each of which does not comprise an electrical contact other than a same common electrical contact shared by the multiple dummy sensing elements.

In an aspect, said determining involves interpolation.

In an aspect, the method further comprises capturing via the exposure a second image with a second radiation detector which comprises a second active area, wherein a shadow of the entire first dummy area with respect to the exposure falls essentially completely on the second active area and intersects the second active area via a shadow active area, and wherein said determining is further based on values of picture elements of the second image corresponding to the shadow active area.

In an aspect, the second radiation detector is bonded to the first radiation detector.

In an aspect, the second radiation detector further comprises a second dummy area disposed between ASIC chips of the second radiation detector.

In an aspect, the first dummy area comprises K straight strips, wherein the second dummy area comprises K straight strips, wherein the K straight strips of the first dummy area and the K straight strips of the second dummy area are parallel to each other, and wherein K is a positive integer.

In an aspect, a thickness of the ASIC chips of the first radiation detector is in a range of 50-100 micrometers.

Disclosed herein is a method, comprising: for i=1, . . . , N, one by one, capturing via an exposure (i) a partial image (1, i) with a same first radiation detector which comprises a first active area and a first dummy area, N being an integer greater than 1; stitching the partial images (1, i), i=1, . . . , N resulting in a first combined image, wherein the first combined image comprises (A) first regular picture elements corresponding to the first active area and (B) first dummy picture elements corresponding to the first dummy area; and determining values of the first dummy picture elements based on values of the first regular picture elements.

In an aspect, the first dummy area comprises K straight strips parallel to a scanning direction of the exposures (i), i=1, . . . , N, and wherein K is a positive integer.

In an aspect, the first dummy area is disposed between application-specific integrated circuit (ASIC) chips of the first radiation detector.

In an aspect, the first dummy area comprises multiple dummy sensing elements each of which comprises an electrical contact which is (A) other than a same common electrical contact shared by the multiple dummy sensing elements, and (B) not electrically connected to the ASIC chips.

In an aspect, the first dummy area comprises multiple dummy sensing elements each of which does not comprise an electrical contact other than a same common electrical contact shared by the multiple dummy sensing elements.

In an aspect, said determining involves interpolation.

In an aspect, the method further comprises: for i=1, . . . , N, one by one, capturing via the exposure (i) a partial image (2, i) with a same second radiation detector which comprises a second active area, wherein a shadow of the entire first dummy area with respect to the exposure (1) falls essentially completely on the second active area and intersects the second active area via a shadow active area; and stitching the partial images (2, i), i=1, . . . , N resulting in a second combined image, wherein said determining is further based on values of picture elements of the second combined image corresponding to the shadow active area.

In an aspect, the second radiation detector is bonded to the first radiation detector.

In an aspect, the second radiation detector further comprises a second dummy area disposed between ASIC chips of the second radiation detector.

In an aspect, the first dummy area comprises K straight strips, wherein the second dummy area comprises K straight strips, wherein the K straight strips of the first dummy area and the K straight strips of the second dummy area are parallel to each other and parallel to a scanning direction of the exposures (i), i=1, . . . , N, and wherein K is a positive integer.

Disclosed herein is a method, comprising: for i=1, . . . , N, one by one, capturing via an exposure (i) a partial image (1, i) with a same first radiation detector which comprises a first active area and a first dummy area, N being an integer greater than 1, wherein the partial image (1, i) comprises (A) regular picture elements (1, i) corresponding to the first active area and (B) dummy picture elements (1, i) corresponding to the first dummy area; for i=1, . . . , N, determining values of the dummy picture elements (1, i) based on values of the regular picture elements (1, i), and assigning the determined values of the dummy picture elements (1, i) to the dummy picture elements (1, i) resulting in a modified partial image (i); and stitching the modified partial images (i), i=1, . . . , N resulting in a first combined image.

In an aspect, the first dummy area is disposed between application-specific integrated circuit (ASIC) chips of the first radiation detector.

In an aspect, the method further comprises, for i=1, . . . , N, one by one, capturing via the exposure (i) a partial image (2, i) with a same second radiation detector which comprises a second active area, wherein a shadow of the entire first dummy area with respect to the exposure (1) falls essentially completely on the second active area and intersects the second active area via a shadow active area, and wherein for i=1, . . . , N, said determining the values of the dummy picture elements (1, i) is further based on values of picture elements of the partial image (2, i) corresponding to the shadow active area.

### BRIEF DESCRIPTION OF FIGURES

FIG. 1 schematically shows a radiation detector, according to an embodiment.

FIG. 2A-FIG. 3C schematically shows different views of the radiation detector, according to different embodiments.

FIG. 4A-FIG. 4D illustrate a first method of imaging, according to an embodiment.

FIG. 5A-FIG. 6C illustrate a second method of imaging, according to an embodiment.

FIG. 7A-FIG. 7I illustrate a third method of imaging, according to an embodiment.

FIG. 8A-FIG. 8B illustrate a fourth method of imaging, according to an embodiment.

FIG. 9 illustrates a fifth method of imaging, according to an embodiment.

FIG. 10A-FIG. 10B show alternative embodiments of the radiation detector.

### DETAILED DESCRIPTION

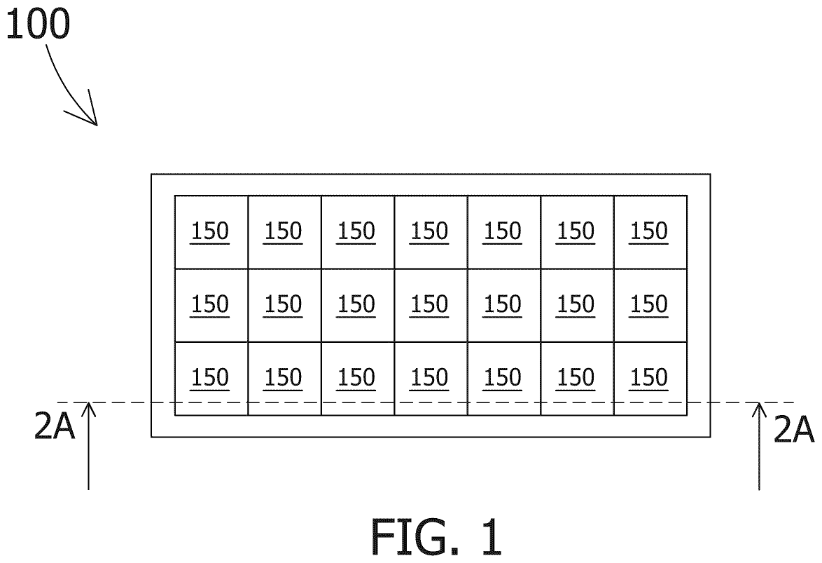

FIG. 1 schematically shows a radiation detector 100, as an example. The radiation detector 100 may include an array of pixels 150 (also referred to as sensing elements 150). The array may be a rectangular array (as shown in FIG. 1), a honeycomb array, a hexagonal array, or any other suitable array. The array of pixels 150 in the example of FIG. 1 has 21 pixels 150 arranged in 3 rows and 7 columns. In general, the array of pixels 150 may have any number of pixels 150 arranged in any way.

A radiation may include particles such as photons (electromagnetic waves) and subatomic particles (e.g., neutrons, protons, electrons, alpha particles, etc.) Each pixel 150 may be configured to detect radiation incident thereon and may be configured to measure a characteristic (e.g., the energy of the particles, the wavelength, and the frequency) of the incident radiation. The measurement results for the pixels 150 of the radiation detector 100 constitute an image of the radiation incident on the pixels. It may be said that the image is of an object or a scene which the incident radiation come from.

Each pixel 150 may be configured to count numbers of particles of radiation incident thereon whose energy falls in a plurality of bins of energy, within a period of time. All the pixels 150 may be configured to count the numbers of particles of radiation incident thereon within a plurality of bins of energy within the same period of time. When the incident particles of radiation have similar energy, the pixels 150 may be simply configured to count numbers of particles of radiation incident thereon within a period of time, without measuring the energy of the individual particles of radiation.

Each pixel 150 may have its own analog-to-digital converter (ADC) configured to digitize an analog signal representing the energy of an incident particle of radiation into a digital signal, or to digitize an analog signal representing the total energy of a plurality of incident particles of radiation into a digital signal. The pixels 150 may be configured to operate in parallel. For example, when one pixel 150 measures an incident particle of radiation, another pixel 150 may be waiting for a particle of radiation to arrive. The pixels 150 may not have to be individually addressable.

The radiation detector 100 described here may have applications such as in an X-ray telescope, X-ray mammography, industrial X-ray defect detection, X-ray microscopy or microradiography, X-ray casting inspection, X-ray non-destructive testing, X-ray weld inspection, X-ray digital subtraction angiography, etc. It may be suitable to use this radiation detector 100 in place of a photographic plate, a photographic film, a PSP plate, an X-ray image intensifier, a scintillator, or another semiconductor X-ray detector.

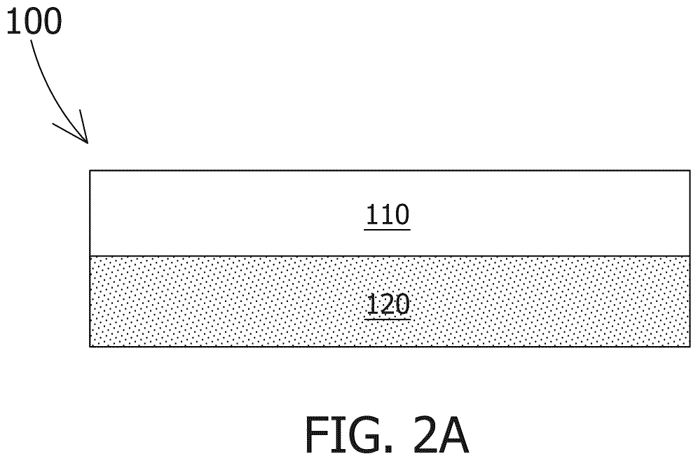

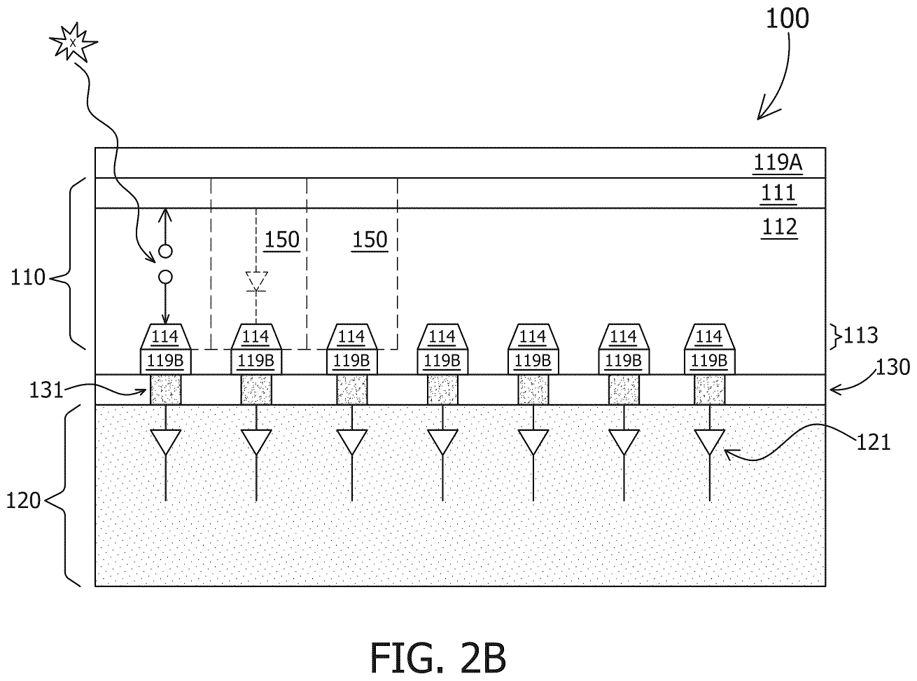

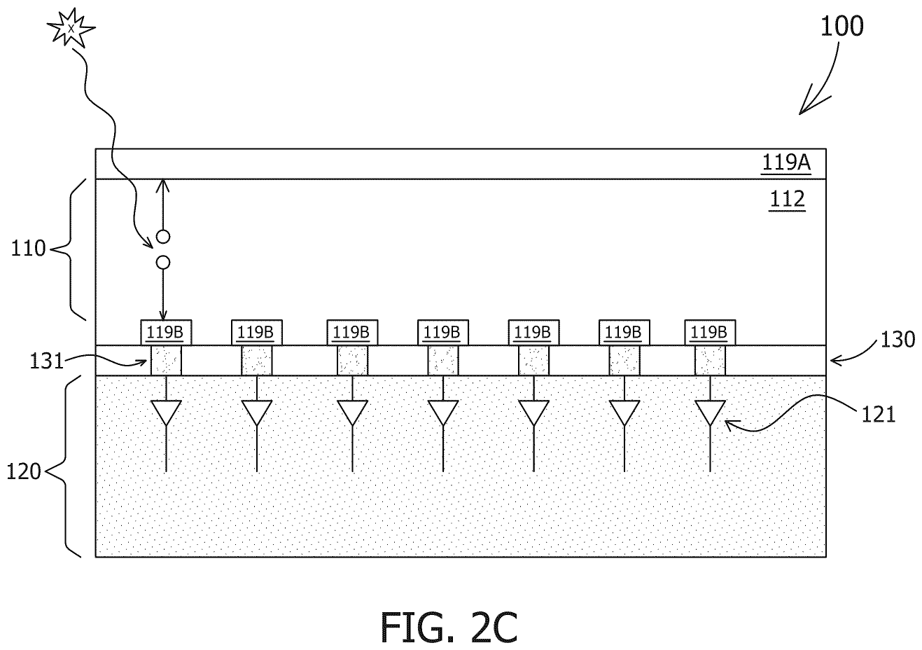

FIG. 2A schematically shows a simplified cross-sectional view of the radiation detector 100 of FIG. 1 along a line 2A-2A, according to an embodiment. More specifically, the radiation detector 100 may include a radiation absorption layer 110 and an electronics layer 120. The electronics layer 120 may include one or more application-specific integrated circuit (ASIC) chips for processing or analyzing electrical signals which incident radiation generates in the radiation absorption layer 110. The radiation detector 100 may or may not include a scintillator (not shown). The radiation absorption layer 110 may comprise a semiconductor material such as silicon, germanium, GaAs, CdTe, CdZnTe, or a combination thereof. The semiconductor material may have a high mass attenuation coefficient for the radiation of interest.

FIG. 2B schematically shows a detailed cross-sectional view of the radiation detector 100 of FIG. 1 along the line 2A-2A, as an example. More specifically, the radiation absorption layer 110 may include one or more diodes (e.g., p-i-n or p-n) formed by a first doped region 111 and one or more discrete regions 114 of a second doped region 113. The second doped region 113 may be separated from the first doped region 111 by an optional intrinsic region 112. The discrete regions 114 are separated from one another by the first doped region 111 or the intrinsic region 112. The first doped region 111 and the second doped region 113 have opposite types of doping (e.g., region 111 is p-type and region 113 is n-type, or region 111 is n-type and region 113 is p-type). In the example of FIG. 2B, each of the discrete regions 114 of the second doped region 113 forms a diode with the first doped region 111 and the optional intrinsic region 112. Namely, in the example in FIG. 2B, the radiation absorption layer 110 has a plurality of diodes (more specifically, FIG. 2B shows 7 diodes corresponding to 7 pixels 150 of one row in the array of FIG. 1, of which only 2 pixels 150 are labeled in FIG. 2B for simplicity). The plurality of diodes have an electrode 119A as a shared (common) electrode. The first doped region 111 may also have discrete portions.

The electronics layer 120 may include an electronic system 121 suitable for processing or interpreting signals generated by the radiation incident on the radiation absorption layer 110. The electronic system 121 may include an analog circuitry such as a filter network, amplifiers, integrators, and comparators, or a digital circuitry such as a microprocessor, and memory. The electronic system 121 may include one or more ADCs. The electronic system 121 may include components shared by the pixels 150 or components dedicated to a single pixel 150. For example, the electronic system 121 may include an amplifier dedicated to each pixel 150 and a microprocessor shared among all the pixels 150. The electronic system 121 may be electrically connected to the pixels 150 by vias 131. Space among the vias may be filled with a filler material 130, which may increase the mechanical stability of the connection of the electronics layer 120 to the radiation absorption layer 110. Other bonding techniques are possible to connect the electronic system 121 to the pixels 150 without using the vias 131.

When radiation from the radiation source (not shown) hits the radiation absorption layer 110 including diodes, particles of the radiation may be absorbed and generate one or more charge carriers (e.g., electrons, holes) by a number of mechanisms. The charge carriers may drift to the electrodes of one of the diodes under an electric field. The field may be an external electric field. The electrical contact 119B may include discrete portions each of which is in electrical contact with the discrete regions 114. The term “electrical contact” may be used interchangeably with the word “electrode.” In an embodiment, the charge carriers may drift in directions such that the charge carriers generated by a single particle of the radiation are not substantially shared by two different discrete regions 114 (“not substantially shared” here means less than 2%, less than 0.5%, less than 0.1%, or less than 0.01% of these charge carriers flow to a different one of the discrete regions 114 than the rest of the charge carriers). Charge carriers generated by a particle of the radiation incident around the footprint of one of these discrete regions 114 are not substantially shared with another of these discrete regions 114. A pixel 150 associated with a discrete region 114 may be a space around the discrete region 114 in which substantially all (more than 98%, more than 99.5%, more than 99.9%, or more than 99.99% of) charge carriers generated by a particle of the radiation incident therein flow to the discrete region 114. Namely, less than 2%, less than 1%, less than 0.1%, or less than 0.01% of these charge carriers flow beyond the pixel 150.

FIG. 2C schematically shows a detailed cross-sectional view of the radiation detector 100 of FIG. 1 along the line 2A-2A, as another example. More specifically, the radiation absorption layer 110 may include a resistor of a semiconductor material such as silicon, germanium, GaAs, CdTe, CdZnTe, or a combination thereof, but does not include a diode. The semiconductor material may have a high mass attenuation coefficient for the radiation of interest. In an embodiment, the electronics layer 120 of FIG. 2C may be similar to the electronics layer 120 of FIG. 2B in terms of structure and function.

When the radiation hits the radiation absorption layer 110 including the resistor but not diodes, it may be absorbed and generate one or more charge carriers by a number of mechanisms. A particle of the radiation may generate 10 to 100,000 charge carriers. The charge carriers may drift to the electrical contacts 119A and 119B under an electric field. The electric field may be an external electric field. The electrical contact 119B includes discrete portions. In an embodiment, the charge carriers may drift in directions such that the charge carriers generated by a single particle of the radiation are not substantially shared by two different discrete portions of the electrical contact 119B (“not substantially shared” here means less than 2%, less than 0.5%, less than 0.1%, or less than 0.01% of these charge carriers flow to a different one of the discrete portions than the rest of the charge carriers). Charge carriers generated by a particle of the radiation incident around the footprint of one of these discrete portions of the electrical contact 119B are not substantially shared with another of these discrete portions of the electrical contact 119B. A pixel 150 associated with a discrete portion of the electrical contact 119B may be a space around the discrete portion in which substantially all (more than 98%, more than 99.5%, more than 99.9% or more than 99.99% of) charge carriers generated by a particle of the radiation incident therein flow to the discrete portion of the electrical contact 119B. Namely, less than 2%, less than 0.5%, less than 0.1%, or less than 0.01% of these charge carriers flow beyond the pixel associated with the one discrete portion of the electrical contact 119B.

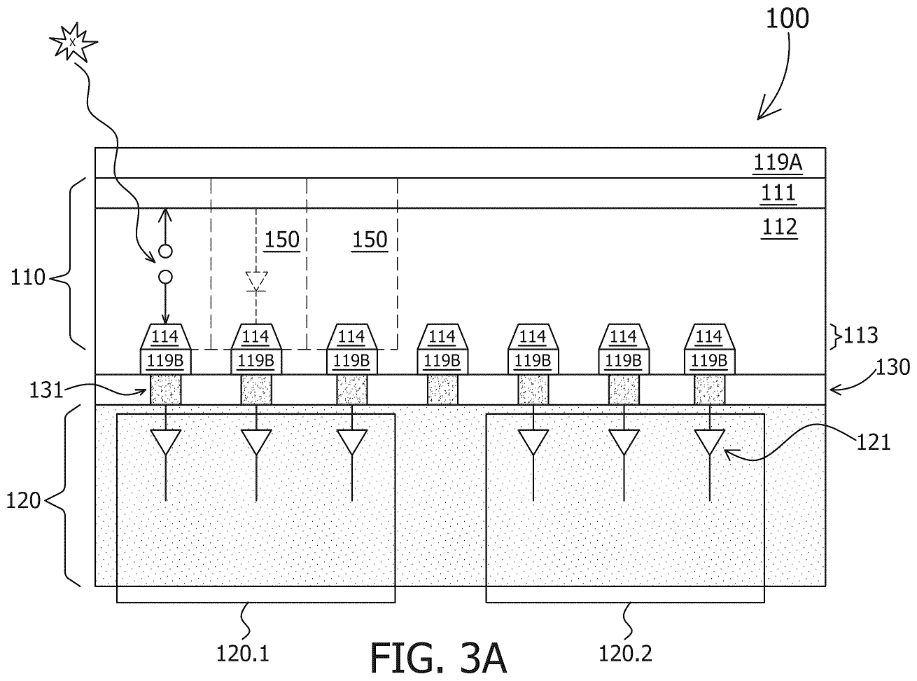

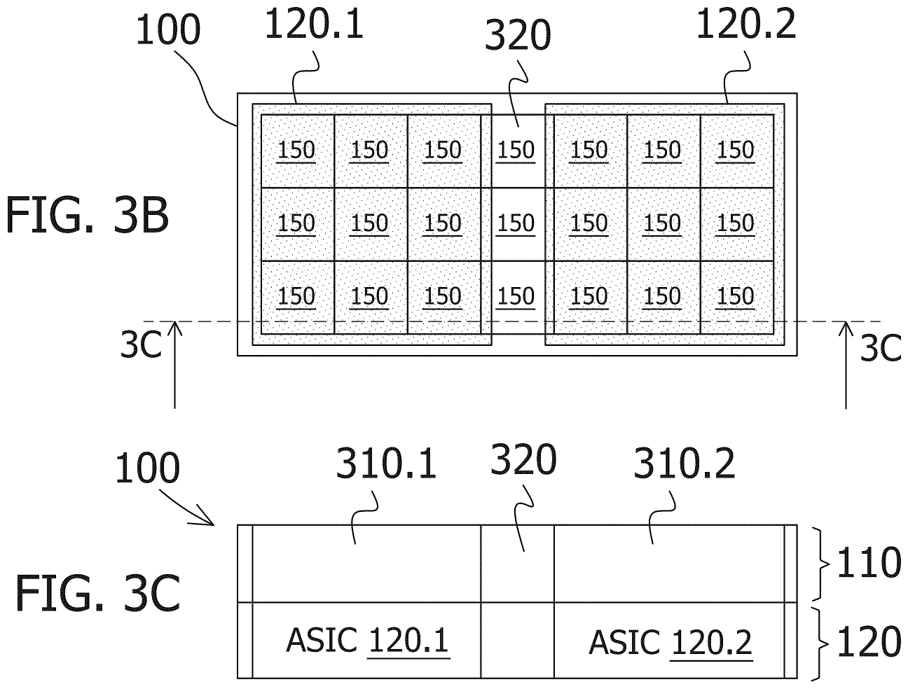

FIG. 3A schematically shows a detailed cross-sectional view of the radiation detector 100 of FIG. 1 along the line 2A-2A, as yet another example. Specifically, the electronics layer 120 may include two ASIC chips 120.1 & 120.2 for processing or analyzing electrical signals which incident radiation generates in the radiation absorption layer 110.

FIG. 3B shows a top view of the radiation detector 100 of FIG. 3A, according to an embodiment. FIG. 3C shows a cross-sectional view of the radiation detector 100 of FIG. 3B along a line 3C-3C, according to an embodiment.

Specifically, in an embodiment, the ASIC chip 120.1 may be for processing or analyzing electrical signals which incident radiation generates in the 9 pixels 150 above the ASIC chip 120.1. Each pixel of the 9 pixels 150 above the ASIC chip 120.1 may be electrically connected to the ASIC chip 120.1. The 9 pixels 150 above the ASIC chip 120.1 form an active region 310.1 (FIG. 3C) which can detect incident radiation.

Similarly, in an embodiment, the ASIC chip 120.2 may be for processing or analyzing electrical signals which incident radiation generates in the 9 pixels 150 above the ASIC chip 120.2. Each pixel of the 9 pixels 150 above the ASIC chip 120.2 may be electrically connected to the ASIC chip 120.2. The 9 pixels 150 above the ASIC chip 120.2 form an active region 310.2 (FIG. 3C) which can detect incident radiation. The active regions 310.1 & 310.2 may be collectively referred to as the active area 310 of the radiation detector 100.

The 3 pixels 150 disposed between the 2 ASIC chips 120.1 & 120.2 (FIG. 3B) may be not electrically connected to the ASIC chips 120.1 & 120.2. As a result, electrical signals which incident radiation generates in these 3 pixels 150 are not received and therefore not processed or analyzed by the ASIC chips 120.1 & 120.2. These 3 pixels 150 may be called dummy pixels or dummy sensing elements. These 3 pixels 150 form a dummy area 320 (FIG. 3B & FIG. 3C) of the radiation detector 100. The dummy area 320 does not detect incident radiation.

In an embodiment, with reference to FIG. 3A & FIG. 3B, each of the 3 dummy pixels 150 (middle of FIG. 3B) may have an electrical contact 119B (see the dummy pixel 150 in middle of FIG. 3A). In an embodiment, this electrical contact 119B is (A) other than the common electrical contact 119A (FIG. 3A) shared by the 3 dummy pixels 150 and (B) not electrically connected to the ASIC chips 120.1 & 120.2 (see FIG. 3A).

In an alternative embodiment, with reference to FIG. 3A & FIG. 3B, each of the 3 dummy pixels 150 (middle of FIG. 3B) may not have the electrical contact 119B (i.e., this electrical contact 119B is not formed for the 3 dummy pixels 150). In other words, each of the 3 dummy pixels 150 does not include an electrical contact other than the common electrical contact 119A (FIG. 3A) shared by the 3 dummy pixels 150.

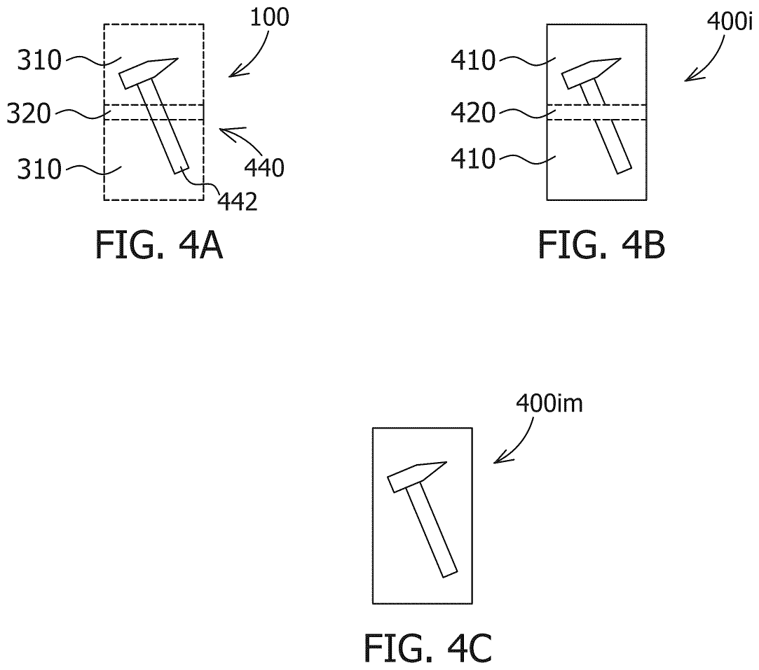

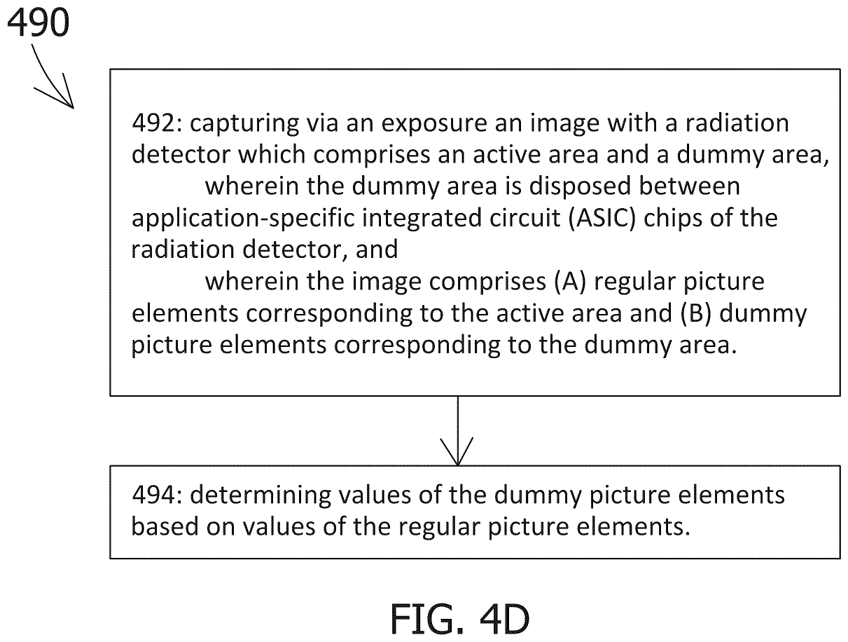

FIG. 4A-FIG. 4C illustrate a first method for obtaining an image of a scene 440 (which includes a hammer 442) with the radiation detector 100, according to an embodiment. For simplicity, in the top view of the radiation detector 100 in FIG. 4A, only the active area 310 and the dummy area 320 of the radiation detector 100 are shown.

In an embodiment, with reference to FIG. 4A, the first method for obtaining an image of the scene 440 may start with an exposure with radiation particles (e.g., X-ray) propagating in a direction perpendicular to the page and going through the hammer 442 and hitting the radiation detector 100 (i.e., from front to behind the page).

As a result of the exposure, the radiation detector 100 may capture an image 400i (FIG. 4B) of the scene 440 which may include (A) regular picture elements 410 corresponding to the active area 310 of the radiation detector 100, and (B) dummy picture elements 420 corresponding to the dummy area 320 of the radiation detector 100. The values of the regular picture elements 410 are related to the scene 440, whereas the values of the dummy picture elements 420 are unrelated to the scene 440 at this time. For example, the values of the dummy picture elements 420 may be arbitrarily set to initial values of zero when the image 400i is created.

Next, in an embodiment, with reference to FIG. 3B, the values of the dummy picture elements 420 may be determined based on the values of the regular picture elements 410. Next, in an embodiment, these determined values may be assigned to the dummy picture elements 420 (thereby replacing their initial values of zero) resulting in a modified image 400im of the scene 440 as shown in FIG. 4C.

In an embodiment, the determination of the values of the dummy picture elements 420 may involve interpolation. Interpolation in this context involves estimating the value of a particular picture element based on the values of the picture elements surrounding that particular picture element.

FIG. 4D shows a flowchart 490 summarizing and generalizing the first method described above. Specifically, in step 492, with reference to FIG. 4A-FIG. 4C, an image (400i) may be captured via an exposure (FIG. 4A) with a radiation detector (100) which comprises an active area (310) and a dummy area (320), wherein the dummy area is disposed between application-specific integrated circuit (ASIC) chips (120.1 & 120.2 in FIG. 3C) of the radiation detector, and wherein the image includes (A) regular picture elements (410) corresponding to the active area and (B) dummy picture elements (420) corresponding to the dummy area. In step 494, the values of the dummy picture elements may be determined based on the values of the regular picture elements.

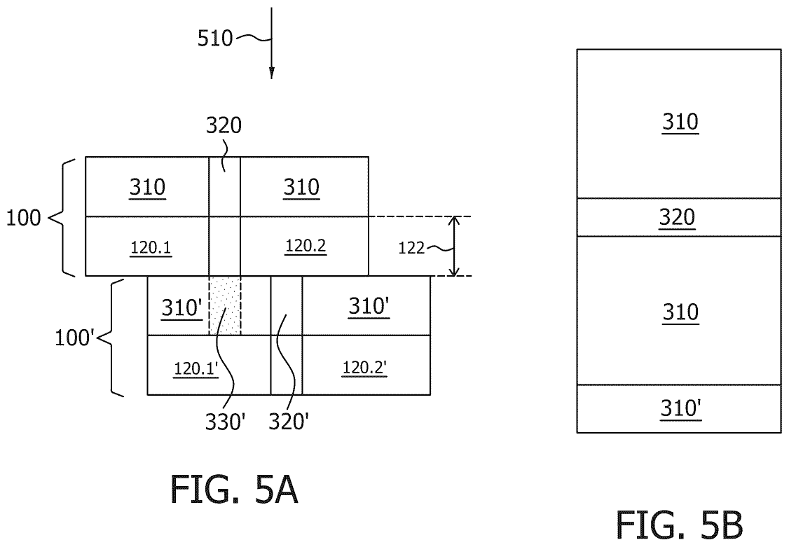

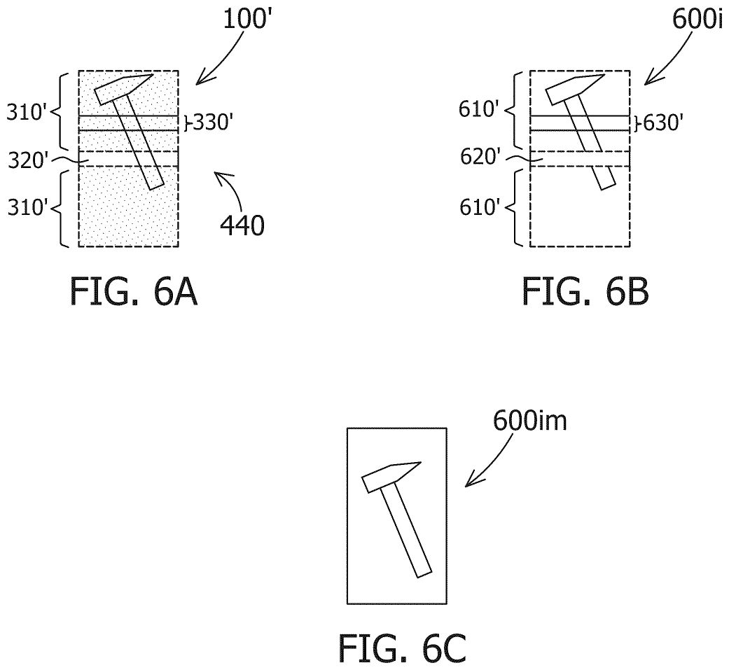

FIG. 5A-FIG. 6C illustrate a second method for obtaining an image of the scene 440 (FIG. 6A), according to an embodiment. In an embodiment, the second method may be an improvement of the first method and may involve the radiation detector 100 and an additional radiation detector 100′ (FIG. 5A). Specifically, the second method may make an improvement to the step 494 (FIG. 4D) of the first method.

In an embodiment, with reference to FIG. 5A, the radiation detector 100′ may be similar to the radiation detector 100. Specifically, the radiation detector 100′ may include an active area 310′, a dummy area 320′, and ASIC chips 120.1′ & 120.2′ which are respectively similar to the active area 310, the dummy area 320, and the ASIC chips 120.1 & 120.2 of the radiation detector 100.

In an embodiment, the dummy area 320′ may be disposed between the ASIC chips 120.1′ & 120.2′. In an embodiment, the dummy areas 320 & 320′ of the radiation detectors 100 & 100′ have the form of 2 straight strips which are parallel to each other.

In an embodiment, with reference to FIG. 5A, the second method may start with the exposure of the first method (i.e., step 492 of FIG. 4D) whose radiation particles propagate in a direction represented by an arrow 510. Reference numeral 510 is hereafter used to indicate the exposure, its radiation particles, and the direction of the radiation particles.

In an embodiment, during the exposure 510, the radiation detector 100′ may be arranged with respect to the radiation detector 100 such that the shadow of the entire dummy area 320 of the radiation detector 100 with respect to the exposure 510 falls essentially completely on the active area 310′ of the radiation detector 100′ (note: “essentially completely” means completely or almost completely). In other words, the radiation detector 100′ is arranged with respect to the radiation detector 100 such that the active area 310′ of the radiation detector 100′ receives essentially all (i.e., all or almost all) the radiation particles of the exposure 510 which have passed through the dummy area 320 of the radiation detector 100.

In an embodiment, a thickness 122 of the ASIC chips 120.1 & 120.2 of the radiation detector 100 may be such that sufficient exposure radiation reaches the radiation detector 100′. In an embodiment, the thickness 122 may be in the range of 50-100 micrometers.

Assume the shadow of the entire dummy area 320 of the radiation detector 100 with respect to the exposure 510 intersects the active area 310′ of the radiation detector 100′ via a shadow active area 330′ (FIG. 5A). FIG. 5B shows a top view of the radiation detectors 100 & 100′ of FIG. 5A.

In an embodiment, the second method may start as follows. During the exposure 510, the radiation detector 100 may capture the image 400i (FIG. 4B) of the scene 440 (FIG. 4A) as in the first method (step 492 of FIG. 4D). Also during the exposure 510, the radiation detector 100′ (FIG. 6A) may capture an image 600i (FIG. 6B) of the scene 440 (FIG. 6A). Next, in an embodiment, the values of the dummy picture elements 420 of the image 400i (FIG. 4B) may be determined not only based on the values of the regular picture elements 410 of the image 400i as in the first method (step 494 of FIG. 4D), but also based on the values of the regular picture elements 630′ (FIG. 6B) of the image 600i corresponding to the shadow active area 330′ (FIG. 5A & FIG. 6A) of the radiation detector 100′.

In an embodiment, the values of the dummy picture elements 420 of the image 400i (FIG. 4B) may be estimated from the values of the regular picture elements 630′ (FIG. 6B) of the image 600i as follows. Assume the average intensity of the image 400i (FIG. 4B) captured by the radiation detector 100 is three times the average intensity of the image 600i (FIG. 6B) captured by the radiation detector 100′. Then, the values of the dummy picture elements 420 of the image 400i (FIG. 4B) may be estimated to be three times the values of the regular picture elements 630′ (FIG. 6B) of the image 600i.

Next, in an embodiment, these determined values may be assigned to the dummy picture elements 420 of the image 400i (FIG. 4B) resulting in a modified image 600im of the scene 440 as shown in FIG. 6C.

In the embodiments described above, the radiation detector 100′ has the dummy area 320′. Alternatively, the radiation detector 100′ may have no dummy area. In an embodiment, the radiation detector 100′ may be bonded to the radiation detector 100 as shown in FIG. 5A. Alternatively, the radiation detector 100′ may be not bonded to the radiation detector 100.

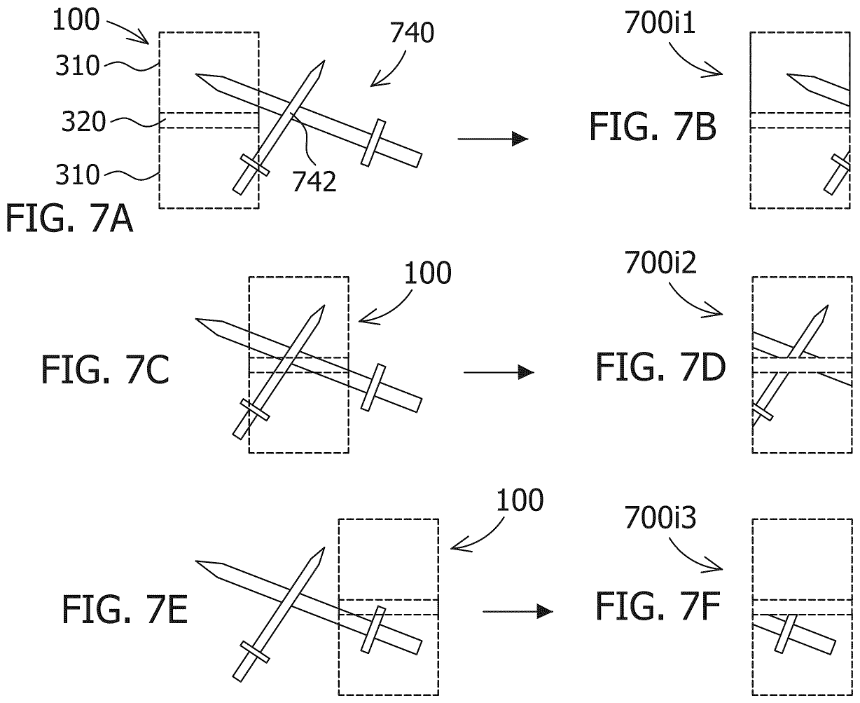

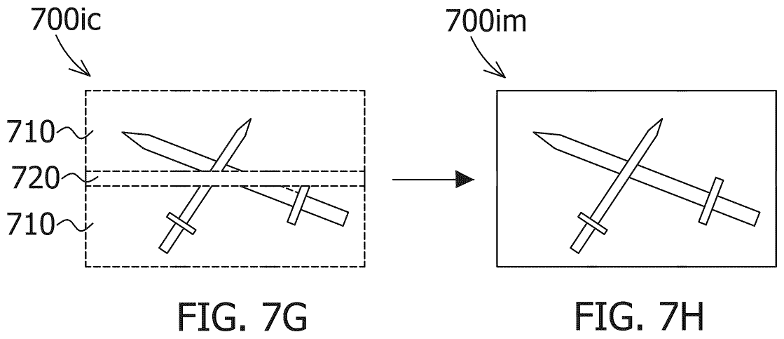

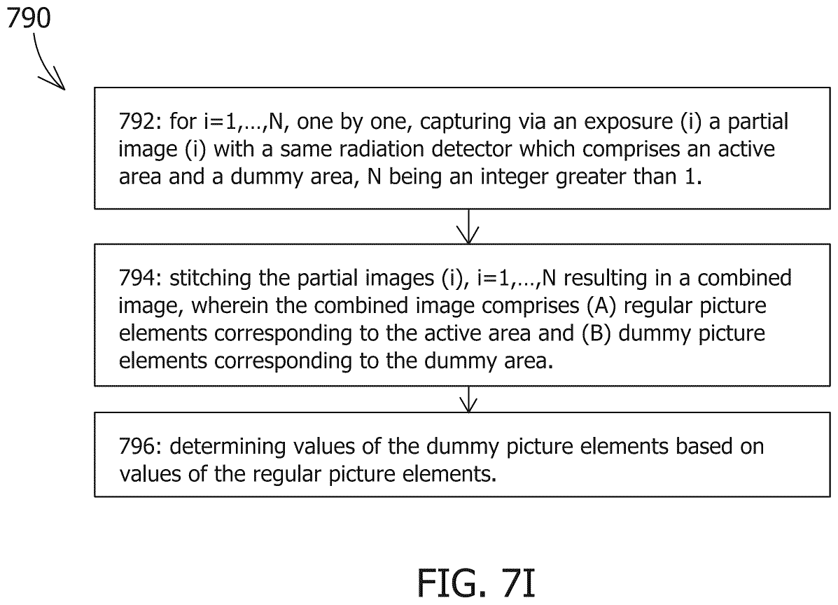

FIG. 7A-FIG. 7H illustrate a third method for obtaining an image of a scene 740 (which includes two swords 742) with the radiation detector 100, according to an embodiment. In an embodiment, the third method may be similar to the first method except that in the third method, multiple exposures and then stitching are performed. Specifically, in an embodiment, the third method may start with a first exposure in which the radiation detector 100 (FIG. 7A) may capture a first partial image 700i1 (FIG. 7B) of the scene 740.

Next, in an embodiment, the radiation detector 100 may be moved horizontally to the right (FIG. 7C) and then a second exposure may be performed in which the radiation detector 100 may capture a second partial image 700i2 (FIG. 7D) of the scene 740. In an embodiment, the movement of the radiation detector 100 between the first and second exposures may be such that the partial images 700i1 & 700i2 overlap each other so as to facilitate later stitching.

Next, in an embodiment, the radiation detector 100 may be moved horizontally further to the right (FIG. 7E) and then a third exposure may be performed in which the radiation detector 100 may capture a third partial image 700i3 (FIG. 7F) of the scene 740. In an embodiment, the movement of the radiation detector 100 between the second and third exposures may be such that the partial images 700i2 & 700i3 overlap each other so as to facilitate later stitching.

Next, in an embodiment, the partial images 700i1, 700i2, and 700i3 may be stitched resulting in a combined image 700ic (FIG. 7G) of the scene 740. The combined image 700ic includes (A) regular picture elements 710 corresponding to the active area 310 of the radiation detector 100, and (B) dummy picture elements 720 corresponding to the dummy area 320 of the radiation detector 100.

Next, in an embodiment, with reference to FIG. 7G, the values of the dummy picture elements 720 of the combined image 700ic may be determined based on the values of the regular picture elements 710. Next, in an embodiment, these determined values may be assigned to the dummy picture elements 720 resulting in a modified image 700im of the scene 440 as shown in FIG. 7H.

In an embodiment, the dummy area 320 of the radiation detector 100 may have the form of a straight strip (FIG. 7A). In an embodiment, the dummy area 320 (having the form of a straight strip) may be parallel to the scanning direction of the first, second, and third exposures. In other words, the radiation detector 100 is arranged such that its dummy area 320 (having the form of a straight strip) is horizontal during the scanning process.

FIG. 7I shows a flowchart 790 summarizing and generalizing the third method described above. Specifically, in step 792, for i=1, . . . , N, one by one, a partial image (i) (e.g., 700i1 in FIG. 7B) may be captured via an exposure (i) (e.g., the first exposure) with a same radiation detector (100 in FIG. 7A) which comprises an active area (310 in FIG. 7A) and a dummy area (320 in FIG. 7A), N being an integer greater than 1 (e.g., N=3 in FIG. 7A-FIG. 7F).

In step 794, the partial images (i), i=1, . . . , N may be stitched resulting in a combined image (700ic in FIG. 7G), wherein the combined image includes (A) regular picture elements (710 in FIG. 7G) corresponding to the active area and (B) dummy picture elements (720 in FIG. 7G) corresponding to the dummy area. In step 796, the values of the dummy picture elements may be determined based on the values of the regular picture elements.

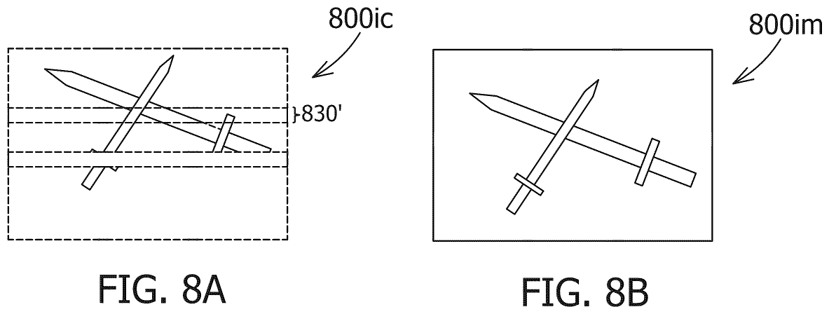

FIG. 8A-FIG. 8B illustrate a fourth method for obtaining an image of the scene 740 (FIG. 7A), according to an embodiment. In an embodiment, the fourth method may be an improvement of the third method described above and may involve the use of both the radiation detectors 100 and 100′ arranged as shown in FIG. 5A. Specifically, the fourth method may make an improvement to the step 796 (FIG. 7I) of the third method.

Specifically, in an embodiment, the fourth method may start with the steps 792 and 794 (FIG. 7I) of the third method. That is the radiation detector 100 may capture the partial images 700i1, 700i2, and 700i3 (FIG. 7B, FIG. 7D, and FIG. 7F) which then may be stitched resulting in the combined image 700ic (FIG. 7G).

Also, during the first, second, and third exposures of the third method, the radiation detector 100′ (FIG. 5A) may capture 3 partial images (not shown) of the scene 740 (FIG. 7A). Next, in an embodiment, the 3 partial images captured by the radiation detector 100′ may be stitched resulting in a combined image 800ic (FIG. 8A) of the scene 740.

Next, in an embodiment, the values of the dummy picture elements 720 of the combined image 700ic (FIG. 7G) may be determined not only based on the values of the regular picture elements 710 of the image 700ic as in the third method (step 796 in FIG. 7I), but also based on the values of the regular picture elements 830′ (FIG. 8A) of the combined image 800ic corresponding to the shadow active area 330′ (FIG. 5A) of the radiation detector 100′.

Next, in an embodiment, these determined values may be assigned to the dummy picture elements 720 of the combined image 700ic (FIG. 7G) resulting in a modified image 800im (FIG. 8B) of the scene 740.

A fifth method for obtaining an image of the scene 740 (FIG. 7A) with the radiation detector 100 may be as follows. In an embodiment, the fifth method may be similar to the first method described above. In the first method, in an exposure, the radiation detector 100 captures the image 400i (FIG. 4B). Then, the values of the dummy picture elements 420 of the captured image 400i are determined and then assigned based on the values of the regular picture elements 410 of the captured image 400i resulting in the modified image 400im (FIG. 4C) of the scene.

In the fifth method, the first method may be repeated multiple times in multiple exposures in a scanning process. For example, the first method may be repeated 3 times with 3 exposures in the scanning process resulting in 3 modified images (not shown) of the scene 740. This scanning process may be similar to the scanning process of the third method described above (FIG. 7A-FIG. 7F). The scanning process of the fifth method may be such that the 3 modified images overlap each other so as to facilitate later stitching. Then, the 3 modified images may be stitched resulting in a combined image (not shown) of the scene 740.

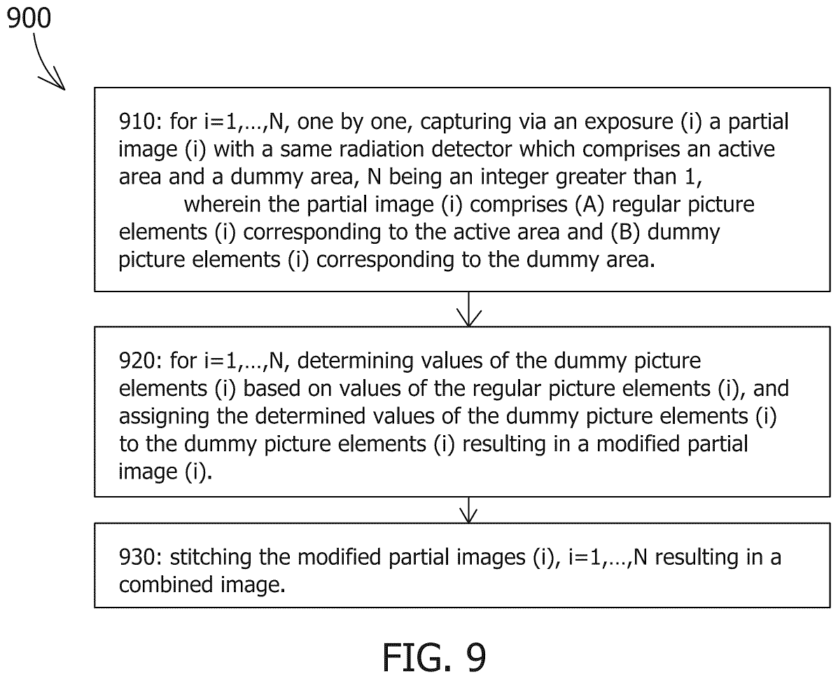

FIG. 9 shows a flowchart 900 summarizing and generalizing the fifth method described above, according to an embodiment. In step 910, for i=1, . . . , N (e.g., N=3), one by one, via an exposure (i) (e.g., the first exposure), a partial image (i) (e.g., image 700i1 of FIG. 7B) may be captured with a same radiation detector (100 of FIG. 7A) which comprises an active area (310) and a dummy area (320), N being an integer greater than 1, wherein the partial image (i) (e.g., the image 700i1 of FIG. 7B) includes (A) regular picture elements (i) corresponding to the active area and (B) dummy picture elements (i) corresponding to the dummy area.

In step 920, for i=1, . . . , N, the values of the dummy picture elements (i) may be determined based on the values of the regular picture elements (i), and these determined values of the dummy picture elements (i) may be assigned to the dummy picture elements (i) resulting in a modified partial image (i). In step 930, the resulting modified partial images (i), i=1, . . . , N may be stitched resulting in a combined image of the scene 740.

A sixth method for obtaining an image of the scene 740 (FIG. 7A) may be as follows, according to an embodiment. In an embodiment, the sixth method may be an improvement of the fifth method and may involve the use of the radiation detectors 100 & 100′ arranged as shown in FIG. 5A. Specifically, the sixth method may make an improvement to the step 920 (FIG. 9) of the fifth method.

Specifically, the sixth method may start with the step 910 of the fifth method (FIG. 9). That is during the 3 exposures, the radiation detector 100 may capture the 3 primary partial images (not shown) of the scene 740. Also during these 3 exposures, the radiation detector 100′ may capture 3 secondary partial images (not shown) of the scene 740.

Next, for each primary partial image of the 3 primary partial images captured by the radiation detector 100, the values of the dummy picture elements of that primary partial image may be determined not only based on the values of the regular picture elements of that primary partial image (as in step 920 in FIG. 9 of the fifth method), but also based on the values of the regular picture elements of the corresponding secondary partial image corresponding the shadow active area 330′ (FIG. 5A) of the radiation detector 100′. Then, the determined values may be assigned to the dummy picture elements of that primary partial image resulting in a corresponding modified primary partial image.

For example, for the first primary partial image of the 3 primary partial images captured by the radiation detector 100, the values of the dummy picture elements of the first primary partial image may be determined not only based on the values of the regular picture elements of the first primary partial image, but also based on the values of the regular picture elements of the first secondary partial image corresponding the shadow active area 330′ (FIG. 5A) of the radiation detector 100′. Then, the determined values may be assigned to the dummy picture elements of the first primary partial image resulting in a first modified primary partial image.

Next, in an embodiment, the step 930 (FIG. 9) of the fifth method may be performed. That is the resulting 3 modified primary partial images may be stitched resulting in a combined image (not shown) of the scene 740. In short, the sixth method makes an improvement to step 920 (FIG. 9) of the fifth method.

In the embodiments described above, each ASIC chip (e.g., 120.1 and 120.2 of FIG. 3C) has the shape of a square (i.e., 3 pixels×3 pixels) and has the size of 9 pixels 150. In general, each ASIC chip may have any shape and size. For example, each ASIC chip may have the shape of a rectangle (e.g., 2 pixels×3 pixels). In general, the ASIC chips do not have to have the same shape and size.

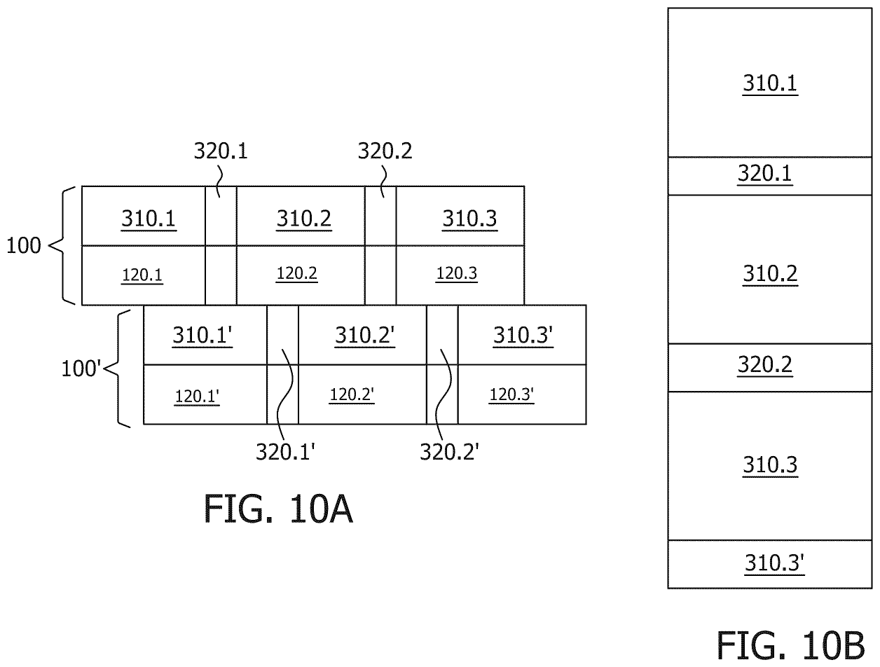

In the embodiments described above, the active area 310 of the radiation detector 100 includes the 2 active regions 310.1 & 310.2 (FIG. 3C). In general, the active area of the radiation detector 100 may have any number of active regions; and the radiation detector 100 may have the same number of ASIC chips. For example, in FIG. 10A, the active area of the radiation detector 100 may include 3 active regions 310.1, 310.2, and 310.3; and the radiation detector 100 may have 3 ASIC chips 120.1, 120.2, and 120.3.

In general, the dummy area of the radiation detector 100 may have any number of dummy regions. For example, in FIG. 10A, the dummy area of the radiation detector 100 may have 2 dummy regions 320.1 and 320.2. In an embodiment, the 2 dummy regions 320.1 and 320.2 may have the form of 2 straight strips which may be parallel to each other as shown in FIG. 10B (a top view of FIG. 10A). In an embodiment, the 2 straight strips may be parallel to the scanning direction of the first, second, and third exposures in the third, fourth, fifth, and sixth methods.

In the embodiments described above (including in FIG. 10A), the radiation detector 100′ is similar to the radiation detector 100. In general, the radiation detector 100′ may be any radiation detector whose physical arrangement with respect to the radiation detector 100 during the exposures is such that essentially all (i.e., all or almost all) exposure radiation particles which have passed through the dummy area 320 of the radiation detector 100 hit the active area of the radiation detector 100′.

In an embodiment, during the exposures described above, a mask (not shown) may be used to block exposure radiation particles that are not aimed at the radiation detectors 100 and 100′. As a result, during the scanning process, in an embodiment, the mask may be moved with the radiation detectors 100 & 100′.

In an embodiment, each of the radiation detectors 100 & 100′ may include a gutter ring on the perimeter that does not detect incident radiation. As a result, if the mask described above is used, then the mask should also block exposure radiation particles that are aimed at the gutter rings of the radiation detectors (in addition to blocking the exposure radiation particles that are not aimed at the radiation detectors).

In an embodiment, the scanning processes described above may be continuous or stepwise. Stepwise scanning means the radiation detector makes a stop to capture an image, and then moves to the next stop to capture the next image, and so on. Continuous scanning means the radiation detector captures images while the radiation detector is moving (no stopping during the scanning).

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method, comprising:
capturing via an exposure a first image with a first radiation detector which comprises a first active area and a first dummy area,
wherein the first dummy area is disposed between application-specific integrated circuit (ASIC) chips of the first radiation detector, and
wherein the first image comprises (A) first regular picture elements corresponding to the first active area and (B) first dummy picture elements corresponding to the first dummy area; and
determining values of the first dummy picture elements based on values of the first regular picture elements;
wherein the first dummy area comprises multiple dummy sensing elements;
wherein each of the multiple dummy sensing elements comprises an electrical contact which is (A) other than a same common electrical contact shared by the multiple dummy sensing elements and (B) not electrically connected to the ASIC chips, or each of the multiple dummy sensing elements does not comprise an electrical contact other than a same common electrical contact shared by the multiple dummy sensing elements.

2. The method of claim 1, further comprising assigning the determined values to the first dummy picture elements.

3. The method of claim 1,
wherein the first dummy area comprises K straight strips parallel to each other, and
wherein K is a positive integer.

4. The method of claim 1, wherein a mask blocks any or almost any radiation particle of the exposure that is (A) not aimed at the first radiation detector or (B) aimed at a gutter ring of the first radiation detector.

5. The method of claim 1, wherein said determining involves interpolation.

6. The method of claim 1, further comprising capturing via the exposure a second image with a second radiation detector which comprises a second active area,
wherein a shadow of the entire first dummy area with respect to the exposure falls essentially completely on the second active area and intersects the second active area via a shadow active area, and
wherein said determining is further based on values of picture elements of the second image corresponding to the shadow active area.

7. The method of claim 6, wherein the second radiation detector is bonded to the first radiation detector.

8. The method of claim 6, wherein the second radiation detector further comprises a second dummy area disposed between ASIC chips of the second radiation detector.

9. The method of claim 8,
wherein the first dummy area comprises K straight strips,
wherein the second dummy area comprises K straight strips,
wherein the K straight strips of the first dummy area and the K straight strips of the second dummy area are parallel to each other, and
wherein K is a positive integer.

10. The method of claim 6, wherein a thickness of the ASIC chips of the first radiation detector is in a range of 50-100 micrometers.

11. A method, comprising:
for i=1, . . . , N, one by one, capturing via an exposure (i) a partial image (1, i) with a same first radiation detector which comprises a first active area and a first dummy area, N being an integer greater than 1;
stitching the partial images (1, i), i=1, . . . , N resulting in a first combined image, wherein the first combined image comprises (A) first regular picture elements corresponding to the first active area and (B) first dummy picture elements corresponding to the first dummy area; and
determining values of the first dummy picture elements based on values of the first regular picture elements;
wherein the first dummy area is disposed between application-specific integrated circuit (ASIC) chips of the first radiation detector;
wherein the first dummy area comprises multiple dummy sensing elements;
wherein each of the multiple dummy sensing elements comprises an electrical contact which is (A) other than a same common electrical contact shared by the multiple dummy sensing elements and (B) not electrically connected to the ASIC chips, or each of the multiple dummy sensing elements does not comprise an electrical contact other than a same common electrical contact shared by the multiple dummy sensing elements.

12. The method of claim 11,
wherein the first dummy area comprises K straight strips parallel to a scanning direction of the exposures (i), i=1, . . . , N, and
wherein K is a positive integer.

13. The method of claim 11, wherein said determining involves interpolation.

14. The method of claim 11, further comprising:
for i=1, . . . , N, one by one, capturing via the exposure (i) a partial image (2, i) with a same second radiation detector which comprises a second active area, wherein a shadow of the entire first dummy area with respect to the exposure (1) falls essentially completely on the second active area and intersects the second active area via a shadow active area; and
stitching the partial images (2, i), i=1, . . . , N resulting in a second combined image, wherein said determining is further based on values of picture elements of the second combined image corresponding to the shadow active area.

15. The method of claim 14, wherein the second radiation detector is bonded to the first radiation detector.

16. The method of claim 14, wherein the second radiation detector further comprises a second dummy area disposed between ASIC chips of the second radiation detector.

17. The method of claim 16, wherein the first dummy area comprises K straight strips,
wherein the second dummy area comprises K straight strips,
wherein the K straight strips of the first dummy area and the K straight strips of the second dummy area are parallel to each other and parallel to a scanning direction of the exposures (i), i=1, . . . , N, and
wherein K is a positive integer.

18. A method, comprising:
for i=1, . . . , N, one by one, capturing via an exposure (i) a partial image (1, i) with a same first radiation detector which comprises a first active area and a first dummy area, N being an integer greater than 1, wherein the partial image (1, i) comprises (A) regular picture elements (1, i) corresponding to the first active area and (B) dummy picture elements (1, i) corresponding to the first dummy area;
for i=1, . . . , N, determining values of the dummy picture elements (1, i) based on values of the regular picture elements (1, i), and assigning the determined values of the dummy picture elements (1, i) to the dummy picture elements (1, i) resulting in a modified partial image (i); and
stitching the modified partial images (i), i=1, . . . , N resulting in a first combined image;
wherein the first dummy area is disposed between application-specific integrated circuit (ASIC) chips of the first radiation detector;
wherein the first dummy area comprises multiple dummy sensing elements;
wherein each of the multiple dummy sensing elements comprises an electrical contact which is (A) other than a same common electrical contact shared by the multiple dummy sensing elements and (B) not electrically connected to the ASIC chips, or each of the multiple dummy sensing elements does not comprise an electrical contact other than a same common electrical contact shared by the multiple dummy sensing elements.

19. The method of claim 18, further comprising, for i=1, . . . , N, one by one, capturing via the exposure (i) a partial image (2, i) with a same second radiation detector which comprises a second active area,
wherein a shadow of the entire first dummy area with respect to the exposure (1) falls essentially completely on the second active area and intersects the second active area via a shadow active area, and
wherein for i=1, . . . , N, said determining the values of the dummy picture elements (1, i) is further based on values of picture elements of the partial image (2, i) corresponding to the shadow active area.
